(19) United States
(12) Patent Application Publication
(10) Pub. No.: US 2009/0182226 A1
(43) Pub. Date: Jul. 16, 2009

(54) CATHETER TRACKING SYSTEM

(76) Inventors: Barry Weitzner, Acton, MA (US); Woojin Lee, Hopkinton, MA (US)

Correspondence Address: VISTA IP LAW GROUP LLP, 12930 Saratoga Avenue, Suite D-2, Saratoga, CA, 95070, US

(21) Appl. No.: 12/409,668

(22) Filed: Mar. 24, 2009

Related U.S. Application Data
(63) Continuation of application No. 10/270,741, filed on Oct. 11, 2002, now abandoned.
Application No. 10/270,741 is a continuation-in-part of application No. 10/216,669, filed on Aug. 8, 2002, now abandoned.
Application No. 10/216,669 is a continuation-in-part of application No. 10/023,024, filed on Nov. 16, 2001, now abandoned.
Application No. 10/023,024 is a continuation-in-part of application No. 10/011,371, filed on Nov. 16, 2001, now Pat. No. 7,090,683.
Application No. 10/011,371 is a continuation-in-part of application No. 10/011,449, filed on Nov. 16, 2001, now abandoned.
Application No. 10/011,449 is a continuation-in-part of application No. 10/010,150, filed on Nov. 16, 2001, now Pat. No. 7,214,230.
Application No. 10/010,150 is a continuation-in-part of application No. 10/022,038, filed on Nov. 16, 2001, now abandoned.
Application No. 10/022,038 is a continuation-in-part of application No. 10/012,586, filed on Nov. 16, 2001, now Pat. No. 7,371,210.
(60) Provisional application No. 60/332,287, filed on Nov. 21, 2001.
(60) Provisional application No. 60/313,496, filed on Aug. 21, 2001.
(60) Provisional application No. 60/269,200, filed on Feb. 15, 2001.
(60) Provisional application No. 60/276,217, filed on Mar. 15, 2001.
(60) Provisional application No. 60/276,086, filed on Mar. 15, 2001.
(60) Provisional application No. 60/276,152, filed on Mar. 15, 2001.
(60) Provisional application No. 60/293,346, filed on May 24, 2001.

(51) Int. Cl. A61B 17/00; A61B 6/12; A61B 19/00

(52) U.S. Cl. 600/424; 606/130

(57) ABSTRACT

A system guides a medical instrument or like implement through an anatomical body such as a human patient. The system may include a drive system which moves the implement through the anatomical body, and a controller that directs the operation of the drive system. The system may also include a plotting system that provides an image of a region of the anatomical body and automatically plots a path for the implement to a site of interest, while the controller directs the drive system to move the implement to the site of interest over the path identified in the image.

### RELATED APPLICATION DATA

This application is a continuation of pending U.S. application Ser. No. 10/270,741 filed Oct. 11, 2002. U.S. application Ser. No. 10/270,741 claims the benefit of U.S. Provisional Application No. 60/332,287 filed Nov. 21, 2001, and is a continuation in part of U.S. application Ser. No. 10/216,669 filed Aug. 8, 2002 (now abandoned), which claims the benefit of U.S. Provisional Application No. 60/313,496 filed Aug. 21, 2001, and is a continuation in part of U.S. application Ser. Nos. 10/023,024 (now abandoned), 10/011,371 (now U.S. Pat. No. 7,090,683), 10/011,449 (now abandoned), 10/010,150 (now U.S. Pat. No. 7,214,230), 10/022,038 (now abandoned), 10/012,586 (now U.S. Pat. No. 7,371,210), all filed Nov. 16, 2001, and all of which claim the benefit of U.S. Provisional Application Nos. 60/269,200 filed Feb. 15, 2001, 60/276,217 filed Mar. 15, 2001, 60/276,086 filed Mar. 15, 2001, 60/276,152 filed Mar. 15, 2001, and 60/293,346 filed May 24, 2001.

The entire teachings of the above applications are incorporated herein by reference.

### BACKGROUND

Catheters are used extensively in the medical field in various types of medical procedures, as well as other invasive procedures. In general, minimally invasive medical procedures involve operating through a natural body opening or orifice of a body lumen, or through small incisions, typically 5 mm to 10 mm in length, through which instruments are inserted. In general, minimally invasive surgery is less traumatic than conventional surgery, due, in part, because no incision is required in certain minimally invasive procedures, or the significant reduction in the incision size in other procedures. Furthermore, hospitalization is reduced and recovery periods are shortened as compared with conventional surgical techniques.

Catheters may be provided in a variety of different shapes and sizes depending upon the particular application. It is typical for a clinician to manipulate the proximal end of the catheter to guide the distal end of the catheter inside the body, for example, through a vein or artery. Because of the small size of the incision or opening and the remote location of the distal end of the catheter, much of the procedure is not directly visible to the clinician. Although clinicians can have visual feedback from the procedure site through the use of a video camera or endoscope inserted into the patient, or through radiological imaging or ultrasonic imaging, the ability to control even relatively simple instruments remains difficult.

In view of the above, some have proposed using robotic tele-surgery to perform minimally invasive procedures. Typically, these robotic systems use arms that reach over the surgical table and manipulate the surgical instruments inserted into the patient, while the surgeon sits at a master station located a distance from the table and issues commands to the arms.

### SUMMARY

The present invention provides a system and method to guide a medical instrument, or like implement, through an anatomical body such as human patient. For example, the implement can be a guide wire or a catheter with an end effector supported at the catheter's distal end. The implement may be guided into the body via an incision, or through a natural body opening or orifice. The system may include a drive system which moves the implement through the anatomical body, and a controller that directs the operation of the drive system.

In some embodiments, the controller enables the drive system to move the implement through the anatomical body while storing data identifying the path of the implement to a site of interest, and subsequently moving the implement to the site of interest lased on the stored data.

In certain embodiments, the system includes a plotting system that provides an image of a region of the anatomical body and automatically plots a path for the implement to the site of interest, while the controller directs the drive system to move the implement to the site of interest over the path identified in the image. The plotting system can digitize the image into digital data that is supplied to the controller so that the controller directs the drive system to move the implement based on the digital data. The plotting system can provide the image in continuous real-time or periodically. The continuous images or updated periodic images enable a user to see how the implement progresses through the anatomical body to the site of interest.

In particular embodiments, the system includes a display that presents the image. The display can include a touch screen which the user touches to identify the location of the site of interest on the screen. In some embodiments, the user uses an input device that interfaces the user with the plotting system to enable the user to select a location corresponding to the site of interest on the image presented on the display. Once the user selects the location of the site of interest, the controller directs the drive system to move the implement from an initial location in the anatomical body to the site of interest no or minimal user intervention. The input device can be a pen or stylus, or a mouse commonly associated with computer systems.

Some embodiments include a tracking system that tracks the movement of the implement as it moves through the body either under automatic or manual control. The controller stores this data to enable it to direct the drive system to move the implement back to this site of interest after having moved the implement to another site of interest.

In particular embodiments, the system includes a force sensing mechanism that enables the implement to feel its way through the anatomical body. In this way, the implement is able to choose a best path of travel, for example, a path of least resistance.

Some embodiments may have one or more of the following advantages. The system frees the clinician from spending time driving the instrument to a particular location in the patient's body. The system no longer requires that the catheter or instrument be directed continuously by an input device. Instead, the surgeon directs the implement by selecting a desired end location or site of interest in the image presented on the display. That is, the surgeon simply tells the instrument to go to that location without any further intervention. Because image can be displayed periodically, exposure of the patient, as well as the surgeon, to X-rays, for example, can be minimized. The clinician can operate the system from a remote location protected from dangerous emissions emanating from the imaging equipment.

The system also has the capability of improving the movement of the implement over that which is directed by a human. In essence, the system is able to move the implement through the body more gently since it employs a computer with a force sensing mechanism to control the implement's movements. Furthermore, with the computer control, a number of different movements may occur to determine a best path of travel for the implement. Also, the system may suggest may suggest multiple possible paths from which the clinician can choose, and/or the clinician can amend the suggested path.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic illustration of a catheter system in accordance with the present invention;

FIG. 2 is a perspective view showing a drive system of the catheter system of FIG. 1;

FIG. 3 is a block diagram of the system of FIGS. 1 and 2;

FIG. 3A is a schematic diagram of a portion of the human anatomy illustrating a catheter and associated guide wire;

FIG. 4 is a schematic illustration of the display of the of FIG. 1;

FIG. 5 is a block diagram illustrating a procedure of using an alterative catheter system in accordance with the invention;

FIG. 6 is a schematic illustration of a body anatomy as it relates to the present invention;

FIG. 7 is a schematic diagram similar to that depicted in FIG. 1 but showing a different form of control by the operator;

FIG. 8 is a flow diagram illustrating a sequence of steps for operating the systems of the present invention; and

FIG. 9 is a flow diagram illustrating an alternative sequence of steps for operating the systems of the present invention.

### DETAILED DESCRIPTION OF THE INVENTION

A description of preferred embodiments of the invention follows.

The present invention described herein, relates to a combination of a robotically controlled instrument or catheter drive system in association with an imaging system that provides images of anatomic structures. The present invention combines these two systems to allow movement instructions to be chosen at the image system for, in turn, controlling the robotic system to move the instrument, catheter, or other implement to a predetermined chosen location. In carrying out the techniques and methods of the present invention it is noted that the operator at the master station is not required to employ continuous band motions to direct the instrument or catheter. Instead, the catheter, instrument, or implement is driven to the desired location, directly and automatically.

Details of a catheter drive system includes as part of a master-slave controller are described in U.S. application Ser. Nos. 10/023,024 (now abandoned), 10/011,449 (now abandoned), 10/022,038 (now abandoned), 10/012,586 (now U.S. Pat. No. 7,371,210), 10/011,371 (now U.S. Pat. No. 7,090,683), and 10/010,150 (now U.S. Pat. No. 7,214,230), all of which were filed Nov. 16, 2001 and are incorporated herein by reference in their entirety. These applications also provide further details regarding the drive system as well as different catheter constructions. Alternatively, articulated arm systems may also be used.

An advantage of the system is that the clinician is free from spending time driving the instrument to a particular location in the anatomy. The system also has the capability of improving the movement of the implement over that which is directed by a human to move the implement more gently, and with the computer control, a number of different movements may occur to determine a best path of travel for the implement. Furthermore, the clinician can operate the system from a remote location that is protected from dangerous emissions, such as x-rays, that are emitted by the imaging equipment.

The imaging system described here is actually a combination of an imaging and tracking system that may be considered as one system, or two separate systems, that is coupled with the catheter drive system. Information about the desired location of the catheter, instrument or implement is inputted into the tracking system and the integrated systems share information to control the catheter drive system to not only plot the path of the implement but also drive the implement to the desired location, and thus move the catheter, instrument or implement to that location. The location corresponds to that selected, for example, by an input device on a display at the imaging system. The imaging may be two-dimensional or three-dimensional.

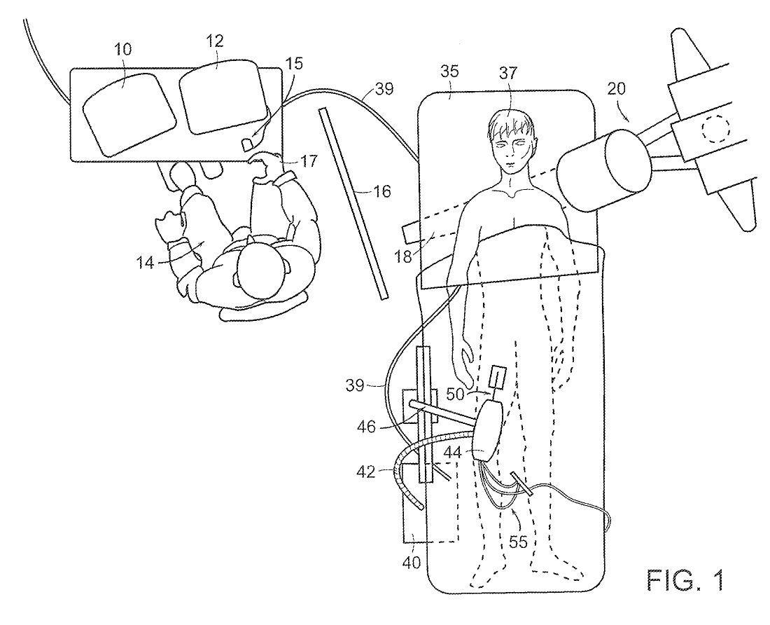

Referring now made to FIGS. 1-4, there is shown a catheter system including a drive system 25 and imaging system 10 that may be a standard biplane angiography system that is used to map coronary arteries. Associated with the imaging system 10 is a display 12 that presents regions of a patient's anatomical body to a clinician such as a surgeon, user or operator 14. These components may be considered as part of the master input station at which an operator 14 is shown seated. Also depicted in FIG. 1 is a lead shield 16 that provides a shield between the operator or clinician 14 and an x-ray beam emanating from a C-arm 18 of the x-ray machine 20, considered as part of the imaging system.

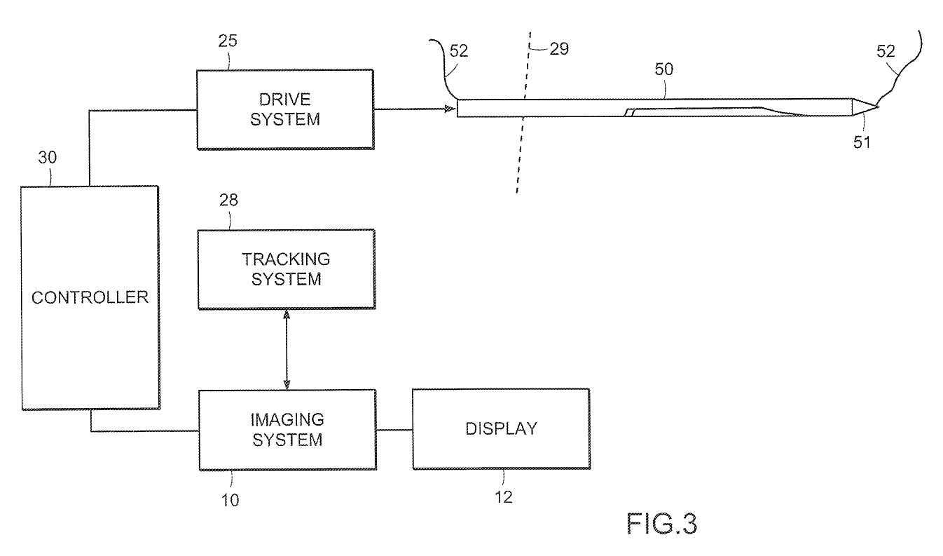

Referring in particular to FIG. 3, the catheter system includes the imaging system 10, the drive system 25, the controller 30, as well as a tracking system 28. Note that the imaging system 10, tracking system 28, and drive system 25 are all intercoupled with the controller 30.

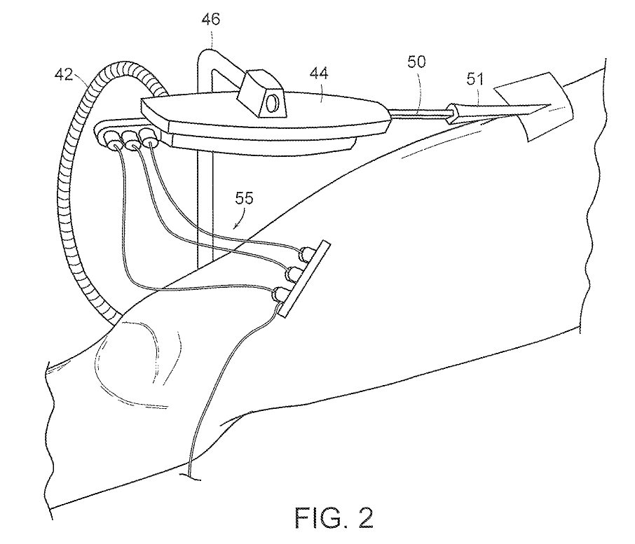

Referring back to FIGS. 1 and 2, there is shown a patient 37 lying on an operating table 35. The tracking system 28 (FIG. 3) and the controller 30 (FIG. 3) may be considered as part of the equipment at the master station as well. Also illustrated in FIG. 1 is electrical cabling 39 that couples to a drive motor array 40. From the motor array 40, there is provided mechanical cabling 42 that couples to the catheter drive member 44. The member 44, such as illustrated in FIG. 2, is supported from a support post 46 that is attached at the site of the table such as schematically illustrated in FIG. 1.

The drive member 44 illustrated in FIGS. 1 and 2 can be considered as part of the drive system 25 shown in the block diagram of FIG. 3. FIGS. 1 and 2 also illustrate the catheter 50 extending from the drive member 44. This is also illustrated in the block diagram of FIG. 3 at catheter 50. In FIG. 3 the catheter 50 can be considered as having an action end 51, which may be similar to the blade 51 illustrated in FIG. 2. The particular catheter illustrated in FIGS. 1 and 2 can be considered as an angioplasty catheter. Associated with such a catheter can also be fluid lines 55 for introducing fluid to the catheter 50.

The technique described in FIGS. 1 and 2 relates to a catheter that is introduced through the femoral vein at the leg of the patient 37. The catheter is of a length to allow it to reach up to the heart. It is noted that the imaging by the x-ray machine 20 is in the area of the heart muscle. Accordingly, this catheter is adapted for control from an initial position at entry of the femoral vein, to a predetermined anatomic site in one of the arteries of the heart.

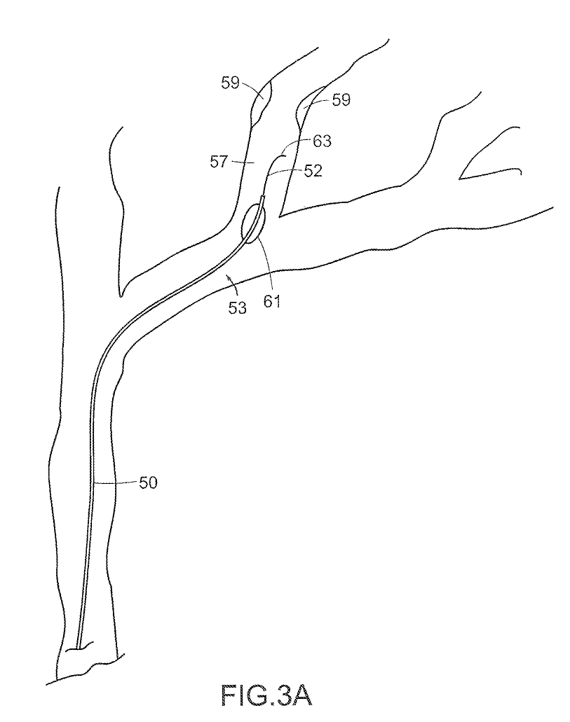

With regard to the tracking system, this may be one of several different commercially available tracking systems. For example, one system is sold by Biosense Webster. These systems are capable of identifying the position of the catheter in, for example, the heart. The concepts of the illustrated embodiments combine such a tracking system with a drive system such as the drive system 25, and described in further detail in the aforementioned applications. A particular feature of the illustrated embodiments is that the system enables the operator or clinician to select a desired location for the catheter. The selection of this location can be by means of, for example, a mouse such as the mouse 15 illustrated in FIG. 1. The operator's hand 17 may operate the mouse to identify on the display 12 a location 21 which is the location where the implement is to be moved from an initial position 23 (FIG. 4). This initial position 23 may correspond, for example, to an incision point of the patient such as illustrated by the dashed line 29 in FIG. 3. Alternatively, as shown in FIG. 4, the operator's hand 17 holds a pen or stylus 9 that can be used to point to the location 21 on the display 12. This technique can be used in association with a touch-screen that will record the particular position selected by the pen or stylus 9.

One application of the concepts presented here can be carried out in connection with a catheter imaging system such as in biplane angiography which is used to map the coronary arteries. In association with such an imaging system, there can be a drive system such as described in the previously mentioned applications as well as the U.S. application filed herewith No. 10/270,740 (now abandoned), and U.S. application filed herewith No. 10/270,743, both of which are incorporated herein by reference in their entirety.

In some implementations, the present invention provides an imaging system coupled with a catheter drive system that can be used to manipulate catheters and guide wires from their proximal ends. For example, manually operable catheters and guide wires can be coupled to the drive system without requiring any modification to the catheter or guide wire. The drive system can be operated by a surgeon at a master station of a master-slave telerobotic system.

In some embodiments, the drive apparatus is in the form of a housing in which the catheter and guide wire are inserted, which are then driven as the surgeon manipulates an input device. In some arrangements, there is a separate drive unit for each of the catheter and guide wire.

An example of an implementation of the catheter drive system of FIG. 3 is shown in FIG. 3A, in which there is illustrated a simple schematic illustration of a part of the human anatomy including an artery or vein 53. The catheter 50 extends through the artery or vein 53 and is shown having its distal end extending into a branch 57 of the artery or vein. For the sake of illustration, the branch 57 is shown as having an obstruction at 59. In the illustrated embodiment, the catheter 50 is shown as having a balloon 61 near its distal end from which the guide wire emerges, and the guide wire 52 has a curved end 63. Thus, the catheter shown in FIG. 3A may be used for a balloon angioplasty procedure. Usually, the guide wire 52 acts as a guide for the catheter 50. That is, the drive system 25 first places the guide wire through the lumen, and then drives the catheter 50 through the lumen along the guide wire 52.

The guide wire 52 as well as the catheter 50 may each be moveable with two degrees-of-freedom. Thus, the drive system 25, under the direction of the surgeon, can move the catheter 50 and guide wire 52 with, for example, both linear and rotational motion. As the guide wire 52 is rotated, the curved end 63 enables the guide wire to be moved through various branches in the artery or vein, for example, the branch 57 is which there is the obstruction 59. As the catheter and guide wire are moved through the body, the surgeon observes their progress through the use of a well-known imaging techniques, such as, for example, Fluoroscopy, CT, Ultrasound, MRI, or PET.

Accordingly, the drive system 25 can control an angioplasty catheter 50 and guide wire 52. The narrowing in the artery that is to be treated is identified by the input device such as the mouse 15 or cursor in association with the angiography image. The drive system 25 places the guide wire 52 in the artery and then moves the angioplasty catheter 50 automatically over the guide wire 52 to the desired location 21. In addition to guidance of angioplasty catheters by angiography, electrophysiologic catheters may be guided by triangulation systems such as those marketed by companies like Biosense Webster and Johnson & Johnson.

As just mentioned, the guide wire 52 can be operated automatically to a predetermined location, or in a simplified version, the guide wire 52 can be inserted manually so that the end of it is at or past the area of blockage. In such a system, the drive system becomes substantially simplified in that it is only required to follow the guide wire to the location selected on the display such as the location 21.

The system shown in FIG. 3 can also be implemented as an angioplasty catheter used in the coronary arteries for removing an obstruction causing a narrowing of an artery. In this regard, the working portion 51 of the catheter 50 is guided to the site of interest identified as the location 21 in FIG. 4. FIG. 1 illustrates a drive member 44 of the drive system 25 driving the catheter and work element through the femoral vein into the proper area in the heart where the surgical activity is to take place.

For further reference to tracking and imaging systems, refer to U.S. Pat. Nos. 6,236,875; 6,246,898; and 6,064,904, all of which are incorporated herein by reference in their entirety.

The imaging and tracking system, once a location such as the location 21 in FIG. 4 has been selected, stores in the controller 30 an image data set, as well as the coordinates of the selected location 21. The controller 30 can include a microprocessor that receives this input information and generates output signals for operating the drive system 25. In the example given, the drive system automatically moves the guide wire 52 through the body lumen to the pre-selected position 21, and then drives the catheter 50 from an initial position 23 to the pre-selected position 21, that is, from an initial start position to a pre-selected final position.

In such a system the algorithm for the control of the drive system 25 is relatively simple. By knowing the initial position 23 and the final position such as the location 21, and furthermore knowing the length of the guide wire 52, the microprocessor in controller 30 simply calculates the distance the catheter 50 is to move. Once this distance is calculated, then the drive system 25 drives the catheter 50 automatically from the initial start position 23 to the pre-selected final location 21.

The initiation of the process can occur with the simple click of a mouse, or by the use of the pen or stylus. Once the process is initiated by the operator, then the drive system operates automatically to transition the guide wire and then the catheter from the initial position to the desired location. While this occurs, the operator may observe on the display the transitional movement of the catheter.

In a simplified version, the guide wire 52 is manually directed to the pre-selected position 21, and the drive system 25 simply automatically drives the catheter 50 substantially linearly along the guide wire 52 from an initial position 23 to a pre-selected position 21.

The use of the tracking system allows the catheter or instrument to know its position, and also the position of other instruments or catheters in the surgical field. For example, at the direction of the surgeon, one implement could be directed to move to another location. Also, points in the surgical field can be selected and labeled, so that the instruments then return automatically at the surgeon's command to these various points.

The system is particularly advantageous in that it no longer requires that the catheter or instrument be directed continuously by an input device. Instead, the surgeon directs the implement by selecting an end desired location; that is, the surgeon simply tells the instrument to go to that location, while the path of the implement is being tracked and optionally observed by the surgeon. The tracking systems described here may be either visual or non-visual and may include endoscopes, digital and analog fluoroscopy, x-ray, CT scanning, ultrasound and MRI.

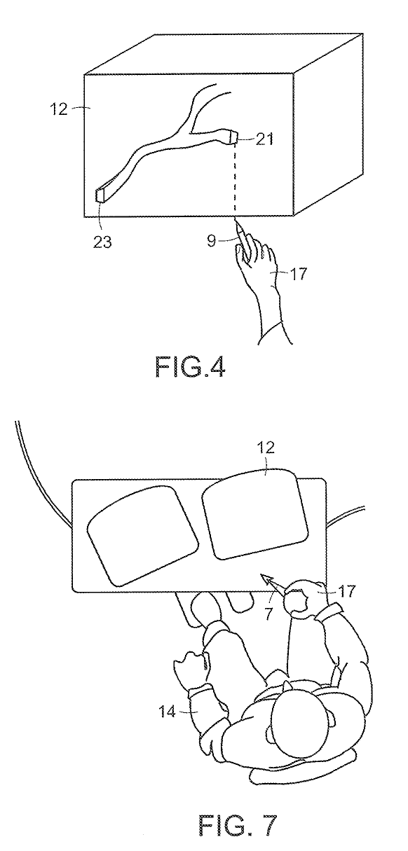

Referring now to FIG. 5, there is shown a block diagram illustrating a particular procedure with an embodiment that slight differs from that described in FIG. 3. The system of FIG. 5 is shown a series of building blocks each of which is, in itself, a known component, for example, a steerable catheter 60, which is well known, and usually manually manipulated by a surgeon.

Another building block of the system of FIG. 5 is illustrates as drive/control system 64. For such a control system refer to U.S. application Ser. Nos. 10/023,024 (now abandoned), 10/011,449 (now abandoned), 10/022,038 (now abandoned), 10/012,586 (now U.S. Pat. No. 7,371,210), 10/011,371 (now U.S. Pat. No. 7,090,683), and 10/010,150 (now U.S. Pat. No. 7,214,230) mentioned previously. Such a system provides for at least one degree-of-freedom of the catheter, usually linear motion thereof and attendant bending or flexing action under control of the system 64.

Also illustrated in FIG. 5 is a block identified as a digital imaging/tracking system 70. The digital imaging/tracking system generates the digital image of the internal anatomic structure. Associated with the digital imaging/tracking system 70 is a display 72, which may be a touch-screen, as described previously in connection with FIG. 3. Thus, the digital imaging/tracking system 70 also records the coordinates of the selected end location to which the catheter is to be driven.

Also identified in FIG. 5 is a machine vision system 75 that is coupled from the digital imaging/tracking system 70. Once the digital imaging/tracking system generates the image data, then, it is the task of the vision system 75 to intelligently decipher the raw data of the pixels and provide an actual “map” of the vessels. The vision system 75 may be of a similar type to that found in systems relating to facial recognition or other types of recognition systems. Similar vision system can also be found in connection with converting topographical maps to actual street locations in connection with geographic mapping concepts.

FIG. 5 also illustrates an intelligent navigation system 78 coupled to the vision system 75. However, outputs from the digital imaging/tracking system 70 may also couple directly to the intelligent navigation system 78. The intelligent navigation system 78 provides the drive signals to the control system 64 for driving the catheter forward and controlling the steering of the catheter.

An output line 73 connecting the digital imaging/tracking system 70 to the machine vision system 75 represents the transmission of the overall pixel images. Also, a line 77 couples the digital imaging/tracking system 70 to the intelligent navigation system 78 to represent the transmission of the co-ordinate information relating to such coordinates as the target position (see location 21 in FIG. 4), the current position, or even an initial position (see location 23 in FIG. 4).

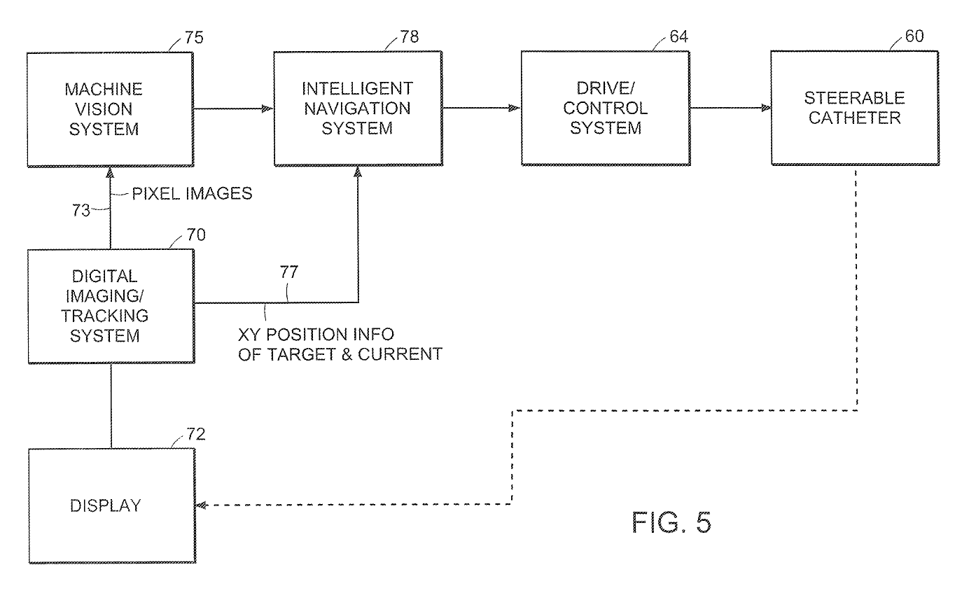

Referring also to FIG. 6, there is shown an example of a vein or artery tree through which the catheter described in FIG. 5 maneuvers, for example, from point A to point B. The catheter may be considered as starting at point A and by the selection on a display, the system provides for automatic transition of the catheter from point A, through a certain vessel path to point B. The points A and B are readily identified in the digital imaging/tracking system 70. The vision system 75 maps the vessel structure, and the intelligent navigation system 78 takes this information and drives the catheter along the path depicted by the dashed line L in FIG. 6.

Also, in connection with the diagram of FIG. 6, the surgeon may desire that the transition from point A to point B occur in smaller increments such as to points A′ and A″. Even when the transition is done in incremental steps, the same type of controls can be applied as illustrated in the block diagram of FIG. 5.

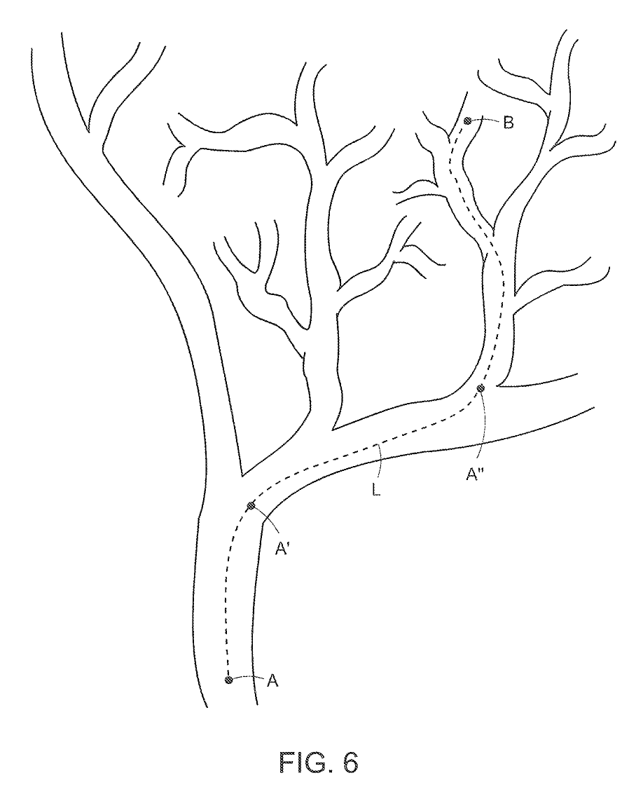

An alternative embodiment of the concepts described here is shown in FIG. 7 that differs somewhat from that described with reference to FIG. 1. The primary difference in the embodiment of FIG. 7 is that the hand 17 of the operator 14 is holding a selection member 7 that is not meant to interact with the display 12 like the mouse 15, but instead points in a particular direction to cause the catheter to transition in the selected direction. Although, the member 7 does not directly interact with the display 12, the member 7 could in fact be a mouse. As such, a left click on the mouse can be used to direct the implement to move forward and a right click used to move it backward.

In another version the member 7 may be in form of a mouse in which the mouse is simple rolled forward to indicate forward advancement of the catheter, and rolled backward for a retreating or backward motion of the catheter.

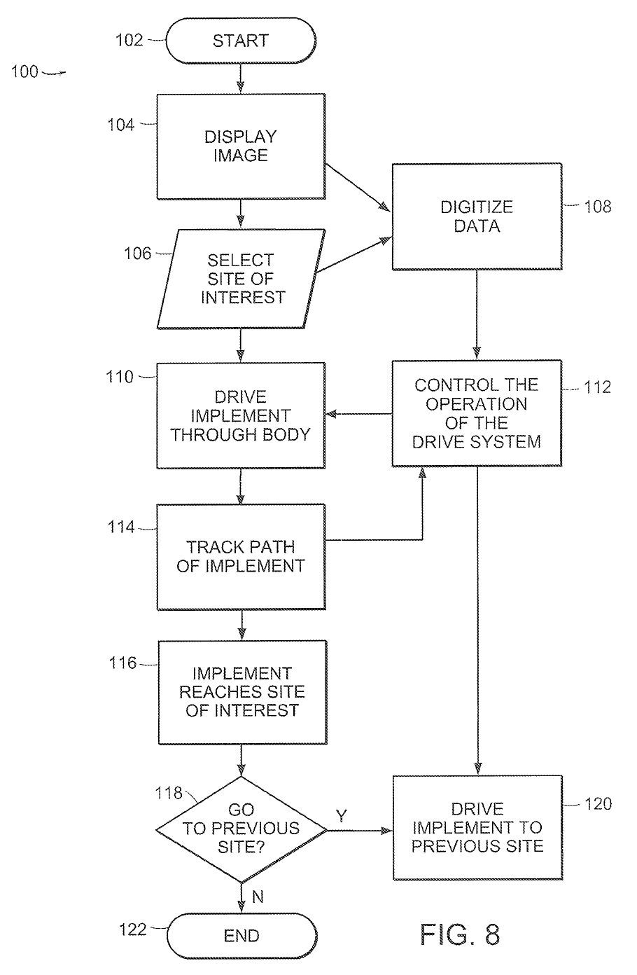

Turning now to FIG. 8, there is shown a process 100 which implements the various embodiments of the systems described above. After initializing the process in a step 102, the imaging system presents an image of a region of the anatomical body on the display 12 in a step 104.

In a step 106, the surgeon or operator uses the input device, such as the pen or stylus 9 or mouse 15, to select a site of interest in the body by identifying the location 21 on the display.

In a step 108, the imaging system 10 digitizes the image along with the initial site 23 and the selected site 21. The controller 30 then in a step 112 uses this digital data to direct the operation of the drive system 25 to move the catheter 50 and implement such as the end effector 51 through the body in a step 110 without any further intervention from the user.

Meanwhile in a step 114 the tracking system 28 tracks the path of the implement 51 as it moves from the initial location 23 through the body, and the controller 30 stores this information in its memory. Note that the imaging system 10 may present the image in continuous real-time as the implement moves along the path, or periodically, in which case, the periodic images are updated to show the progress of the implement. An advantage of periodic imaging is that exposure of the patient, as well as the surgeon, to X-rays, for example, can be minimized.

In a step 116 the implement finally reaches the site of interest 21. In a decision step 118, the user decides if the implement 51 is to return to a previously identified site of interest. If the user wishes not to return to a previous site, the process 100 ends in a step 122. However, if the user wants the implement to be moved to a previously identified site, then in a step 120 the drive system 25 moves the implement 51 to that site under the direction of the controller 30, which uses the tracking data obtained in the step 114 stored in the controller's memory.

A particular feature of the system is that the drive system 25 and controller 30 have a force sensing mechanism that allows the implement 51 to feel its way through a lumen or body. In this way, the implement 51 moves along a best path of travel, for example, a path of least resistance.

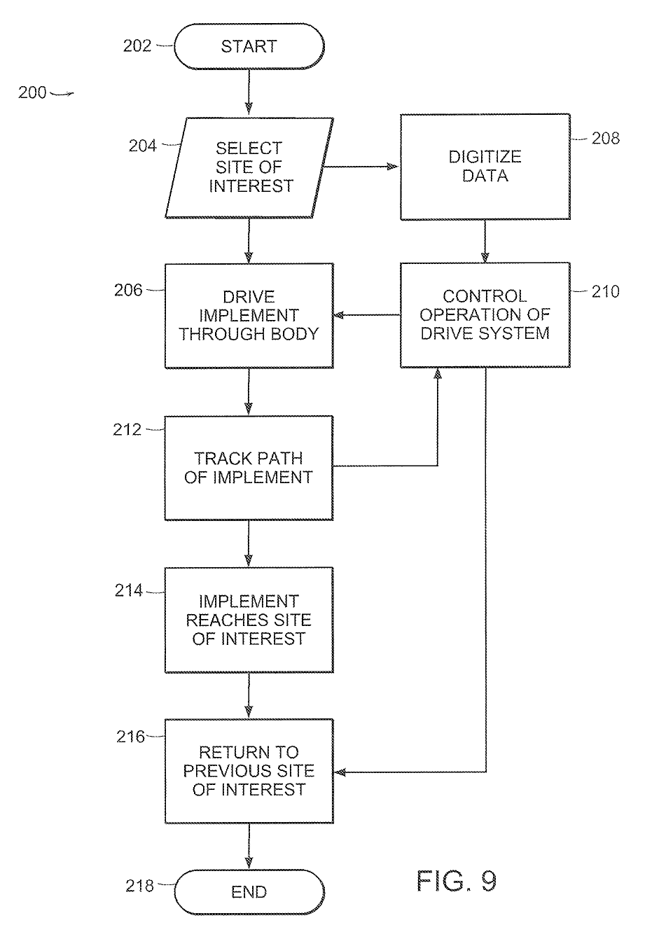

Referring now to FIG. 9, there is shown an alternative process 200 for guiding an implement 51 through an anatomical body. Similar to the process 100, the user selects a site of interest in a step 204. This information is then digitized in a step 208, and the controller 30 uses this digital data in a step 210 to direct the operation of the drive system 25.

Under the direction of the controller 30, the drive system 25 moves the implement 51 to the site of interest in a step 206, while the tracking system 28 tracks the path of the implement in a step 212. After the implement reaches the site of interest in a step 214, the controller 30 subsequently directs the drive system 25 to move the implement to a previous site of interest. The implement moves along a path to this previous site based on data stored in the controller 30 as the implement was tracked in a previous step 212.

Like the process 100, the process 200 can implement a force sensing mechanism that enables the implement to move through the anatomical body along a best path of travel such as a path of least resistance. The process 200 can also implement an imaging system which displays a region of the anatomical body, in which case, the surgeon or operator can select the site of interest through the use of an input devices such as a pen, stylus, or mouse.

A relatively simply algorithm has been described above for essentially advancing the catheter from an initial position to a final position, essentially linearly through an anatomical body. Other more complex algorithms are also contemplated as falling within the scope of the present invention, and which can take into account other more complex paths that the catheter may be expected to transition such as illustrated in FIG. 6. Although the system specifically described above relates to catheters, the principles and concepts also apply to control of other instruments or implements. For example, in laparoscopic or other types of surgery the technique can be used to transition an instrument automatically from an initial position, such as where the instrument is initially placed upon insertion by a surgeon through an incision, to a final position at which some predetermined surgical procedure is to take place. For such procedures for instruments the control algorithm can also be quite simple because the transition from start to final positions may be considered as a direct linear transition, or may be an algorithm that addresses a circuitous path such as illustrated in FIG. 6.

Furthermore, there have been described herein the selection of a final implement position by use of a pen or mouse in association with a display. Also covered by the invention, however, would be other selection concepts. For example, rather than direct contact with a display, a location could be identified by its coordinates, and a coordinate number could be entered into the system such as through a keyboard. Once entered the operator could then hit an “execute” key to initiate the transition of the implement to the selected final position.

This invention can be implemented and combined with other applications, systems, and apparatuses, for example, those discussed in greater detail in U.S. Provisional Application No. 60/332,287, filed Nov. 21, 2001, the entire contents of which are incorporated herein by reference, as well as those discussed in greater detail in each of the following documents, all of which are incorporated herein by reference in their entirety:

U.S. application Ser. No. 09/783,637 filed Feb. 14, 2001 (now abandoned), which is a continuation of PCT application Serial No. PCT/US00/12553 filed May 9, 2000, which claims benefit of U.S. Provisional Application No. 60/133,407 filed May 10, 1999; U.S. application Ser. No. 10/208,087 filed Jul. 29, 2002 (now abandoned), which is a continuation of U.S. application Ser. No. 09/827,503 filed Apr. 6, 2001 (now U.S. Pat. No. 6,432,112), which is a continuation of U.S. application Ser. No. 09/746,853 filed Dec. 21, 2000 (now U.S. Pat. No. 6,692,485), which is a divisional of U.S. application Ser. No. 09/375,666 filed Aug. 17, 1999, (now U.S. Pat. No. 6,197,017), which is a continuation of U.S. application Ser. No. 09/028,550 filed Feb. 24, 1998 (now abandoned); PCT application Serial No. PCT/US01/1 1376 filed Apr. 6, 2001, which claims priority to U.S. application Ser. No. 09/746,853 filed Dec. 21, 2000 (now U.S. Pat. No. 6,692,485), and U.S. application Ser. No. 09/827,503 filed Apr. 6, 2001 (now U.S. Pat. No. 6,432,112); U.S. application Ser. Nos. 10/014,143 (now abandoned), 10/012,845 (now U.S. Pat. No. 7,169,141), 10/008,964 (now abandoned), 10/013,046 (now abandoned), 10/011,450 (now abandoned), 10/008,457 (now U.S. Pat. No. 6,949,106), and 10/008,871 (now U.S. Pat. No. 6,843,793), all filed Nov. 16, 2001 and all of which claim benefit to U.S. Provisional Application No. 60/279,087 filed Mar. 27, 2001; U.S. application Ser. No. 10/077,233 filed Feb. 15, 2002 (now U.S. Pat. No.7,297,142), which claims the benefit of U.S. Provisional Application No. 60/269,203 filed Feb. 15, 2001; U.S. application Ser. No.10/097,923 filed Mar. 15, 2002 (now U.S. Pat. No.6,860,878), which claims the benefit of U.S. Provisional Application No. 60/276,151 filed Mar. 15, 2001; U.S. application Ser. No. 10/034,871 filed Dec. 21, 2001 (now U.S. Pat. No.6,810,281), which claims the benefit of U.S. Provisional Application No. 60/257,816 filed Dec. 21, 2000; U.S. application Ser. No. 09/827,643 filed Apr. 6, 2001 (now U.S. Pat. No. 6,554,844), which claims the benefit of U.S. Provisional Application No. 60/257,869 filed Dec. 21, 2000, and U.S. Provisional Application No. 60/195,264 filed Apr. 7, 2000.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for guiding a distal portion of a flexible medical instrument through an interior region of an anatomical body, comprising:
mechanically driving a proximal end of the instrument to thereby initiate movement of a distal end portion of the instrument within the anatomical body;
storing position data representing a trajectory taken by the distal end portion of the instrument as it is being moved within the anatomical body; and
subsequently and automatically directing the mechanically driving of the proximal end of the instrument based at least in part upon the stored position data to thereby move the distal end portion of the instrument to a site of interest within the anatomical body.

2. The method of claim 1, further comprising interfacing a user with the implement.

3. The method of claim 2, further comprising presenting on a display an image of a region of the anatomical body to the user.

4. The method of claim 3, further comprising operator-selection of the site of interest based on the displayed image.

5. The method of claim 4, wherein the display comprises a touch-screen user input interface, the method further comprising operator-selection of the site of interest by touching the location of the site of interest on the display.

6. The method of claim 3, wherein the presented image is updated in continuous real-time to show the progress of the distal end portion of the instrument as it moves to the site of interest.

7. The method of claim 3, wherein the presented image is periodically updated to show the progress of the distal end portion of the instrument as it moves to the site of interest.

8. The method of claim 1, wherein the trajectory is along a path of least resistance within the anatomical body.
